(19) United States
(12) Patent Application Publication
(10) Pub. No.: US 2022/0015791 A1
(43) Pub. Date: Jan. 20, 2022

(54) AN APPARATUS FOR HARVESTING A FASCIA LATA GRAFT

(71) Applicant: ISTANBUL MEDIPOL UNIVERSITESI, Istanbul (TR)

(72) Inventor: Necdet ÖZCELIK, Istanbul (TR)

(21) Appl. No.: 17/311,456

(22) PCT Filed: Dec. 24, 2019
(86) PCT No.: PCT/TR2019/051193

§ 371 Date: Jun. 7, 2021

(30) Foreign Application Priority Data
Dec. 25, 2018 (TR) 2018/20459

(51) Int. Cl. A61B 17/3205

(52) U.S. Cl. CPC A61B 17/3205; A61B 2017/320052

(57) ABSTRACT

A fascia lata graft harvesting apparatus that is intended to be used in surgical interventions and that allows the fascia to be harvested safely. The apparatus includes a metal part shaped like a guide strip placed under the fascia lata, a cutting tool which enables to free the graft by the shearing effect created by the end part thereof, and a clamp which fixes the fascia on the guide metal strip, thereby preventing it from sliding and at the same time ensuring that the guide metal strip is kept in position.

### TECHNICAL FIELD

The present invention relates to a medical appliance that is used in surgical interventions.

The invention, in particular, relates to an appliance for harvesting a fascia lata graft that comprises a guide metal in strip form which is placed under the fascia lata allowing the fascia to be harvested safely and helping the graft to be released with a shearing effect it produces at its end portion.

### PRIOR ART

Graft (patch) is the process of harvesting any tissue in the body to be grafted from one site to another site without any vascular and neural connections feeding said tissue. It is not possible for this tissue to be fed after subjecting it to vascular repair at the body site to where it is transplanted. These tissues feed themselves by the use of a method called “diffusion” from proximal tissues and capillary veins thereof and new vascularization processes are observed in time. To eliminate feeding problems, grafts are preferred to be thin and to be used in small amounts; ultimately it is aimed to ensure that they attach themselves to the new site and are accepted at the new site where they are transplanted in the body. A variety of suture materials are used in increasing and repairing tension of some sites in the body. However, an autologous material (autologous=one's own tissue) that is prepared from one's own tissue and can be used in place of suture materials is preferable as it is accepted by the body and it provides more favorable outcomes. The most commonly used graft tissues are; skin, cartilage, bone, vein, nerve, fascia, fat, dermis, hair follicle, nail bed, and root cell.

The fascia is fibrous sheets which are grey-white colored and are formed of connective tissue covering muscles and muscle groups. They have a structure formed of histological collagen, elastane and fibroblast. They have no neural or vascular characteristics. They form a basis for muscles to attach to the related site at origo (starting) and insertio (ending) points. Moreover, they also shape the half ring-shaped connective tissue that allows tendons to bind to the site where they are located. Fascia lata is the continuation of fascia glutea. The fascia covers the muscles located at the outer side of the femur. It has been given this name as it is long and wide. The section of the fascia lata that covers musculus quadriceps femoris is loose and it is usually confused with the aponeurosis of musculus tensor fascia latae. Its upper boundary is not distinct and it continues with fascia glutealis in the gluteal region. It ends after merging with musculus gluteus superficialis

Fascia lata grafts are easy to obtain and prepare. In addition to this; they are a biological material that is increasingly being used due to their characteristics of having high tensile strength and being convenient for suture applications, being well adaptable to the site to which they are applied as grafts by attaching to the site for a long time, being easy to manipulate during an operation, having low risk of postoperative complication and being capable of preventing adhesion formation.

Fascia lata grafts are successfully used in a great variety of specific cases including; repair of abdominal hernias, ptosis treatment, repair of scleral defects, urogenital system surgery, orthopedic surgery, chest wall reconstruction, operative treatment of fecal incontinence, brain and nerve surgery, repair of heart and vascular valve insufficiencies, facial paralysis, diaphragmatic hernia, urethral and penile reconstruction.

Materials for the harvesting of fascia lata grafts used today are not capable of harvesting healthy grafts that are always usable. If grafts are not harvested properly, they are not capable of providing favorable outcomes in the site to which they are applied. As it is not always possible to obtain usable fascia in systems where several incisions should not be made and the process is carried out subcutaneously in the prior art, and as the same side can be tried again for grafting following an unsuccessful application, will cause the muscle sheath to be more open and could cause muscle hernia and the other leg of the patient will have to be used. In such cases, it causes the patient to have problems following surgery and it prolongs the healing process.

In conclusion, the necessity to provide a novelty in the related technical field has risen due to the aforementioned drawbacks and the ineffectiveness of the current solutions regarding the subject matter.

### BRIEF DESCRIPTION OF THE INVENTION

The present invention is related to an apparatus for harvesting fascia lata graft, which meets the aforementioned requirements, eliminates all of the disadvantages and provides some additional advantages.

The primary aim of the invention is to ensure that the fascia is harvested in a strip shape and the graft is released with a shearing effect at its end portion without traumatizing proximal tissue by securing the fascia lata with a strip-shaped metal guide, which is placed beneath the fascia lata.

An aim of the invention is to ensure that the last 1 mm portion of the strip made of rigid material such that it can be inserted and moved under the fascia at the thigh along the fascia is bent to be suitable to perform a cutting process in order to free the fascia by creating a guillotine effect, and that the end sections of the strip is rounded out so that it does not damage proximal tissues.

Another aim of the invention is to provide a harvesting process of fascia lata graft without performing a second cutting process.

Another aim of the invention is to ensure healthy and usable graft harvesting in a single step. This has a good impact on the post-operation healing process of a patient.

Another aim of the invention is to ensure that the fascia can be harvested at any desired length by means of the score lines on the guide strip. Thus, it will be possible to meet the need for harvesting grafts at different lengths with a single apparatus without the need to produce another apparatus that has a different length.

Another aim of the invention is to form a notch similar to a drawer rail at the entrance section of a duct in order to ensure that the guide strip does not change its place or slide up-down while being moved through the cutting tool, and to ensure that the guide strip moves backward or forward from the lower level inside the cutting tool and does not damage the graft by shifting upward. It also aims to provide a guide path to ensure the guide strip is accurately placed inside the duct at the lower level and that it moves smoothly.

In an effort to achieve the aforementioned aims, the present invention is related to an apparatus for harvesting a fascia lata graft which is intended to be used in surgical interventions, comprising; a guide and fascia protecting strip made of a rigid material such that it can be moved from under the fascia in the thigh along the whole fascia, bent and rounded end sections, that are suitable to perform a cutting process at the last 1 mm section of the strip in order to free the fascia by creating a guillotine effect, a cutting tool that enables the fascia to be completely freed from proximal tissues, which is wide enough to allow the guide and the fascia protecting strip to pass through, and a clamp that fastens the fascia on the metal guide strip, prevents it from sliding and helps to keep the metal guide strip in position.

The invention can also be used for harvesting a strip-shaped tissue without damaging proximal tissues in cases such as robotic surgeries that are applied in narrow and closed areas.

All of the structural and characteristic features and advantages of the invention will be better understood by means of the accompanying drawings and the detailed description presented with reference to these drawings and therefore if an evaluation is to be carried out, these drawings and the detailed description should be taken into consideration.

### FIGURES OF THE INVENTION

FIG. 1, is an overall view of the appliance for harvesting fascia lata graft

FIG. 2, is a side view of the guide and fascia lata protecting strip.

FIG. 3, is a side view of the fascia cutting tool.

FIG. 4, is an upper view of the guide and fascia lata protecting strip.

FIG. 5, is a front view of the fascia cutting tool.

FIG. 6, is a front view of the fascia fastening clamp.

FIG. 7, is a rear view of the fascia fastening clamp.

The drawings do not need to be scaled and the details that are not necessary to understand the present invention may have been omitted. Furthermore, the elements that are at least substantially identical or the elements that have at least substantially identical functions are indicated with the same reference numbers.

### DEFINITIONS OF THE REFERENCE NUMBERS

10. Fascia lata graft harvesting appliance
20. Guide and Fascia protecting strip
   21. Score line
   22. Rounded end
   23. Uneven surface
30. Fascia cutting tool
   31. Cutting tool
   32. Separator head portion
   33. Cutting end portion
   34. Duct
40. Fascia fastening clamp
   41. Clamp/release bolt with an anti-sliding surface
   42. Fascia fastening foot portion
   43. Clamp frame for placing in the guide metal strip

### DETAILED DESCRIPTION OF THE INVENTION

In this detailed description, the apparatus for harvesting fascia lata subject to the present invention and the preferred embodiments thereof are disclosed only to provide a better understanding of the subject matter, such that they do not limit the scope of the invention.

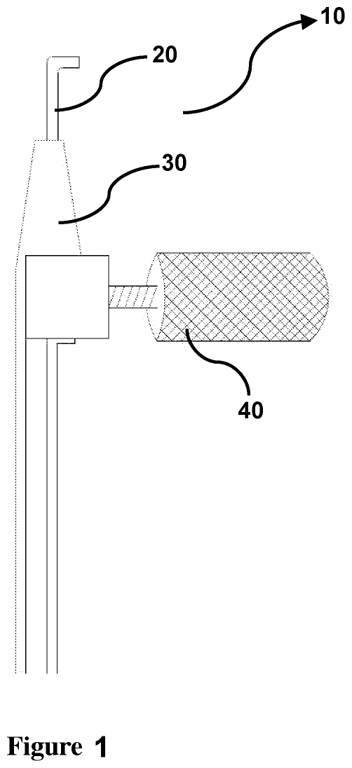

A general view of the fascia lata graft harvesting apparatus (10) that has been developed to be used in surgical interventions, in order to ensure safe and accurate harvesting of fascia has been illustrated in FIG. 1. The graft harvesting apparatus (10) is basically formed of three individual parts; the guide and fascia protecting metal strip (20), the fascia cutting tool (30) and the clamp (40) that is used for preventing the fascia from sliding, and that allows the guide and fascia protecting metal strip (20) to be kept in a fixed position. The strip (20) and the cutting tool (30) are positioned to be interlocked, the clamp (40) prevents the fascia from sliding whilst allowing the metal strip to be kept in fixed position, and the harvesting application is conducted when the fascia lata graft harvesting apparatus (10) is in its integrated state.

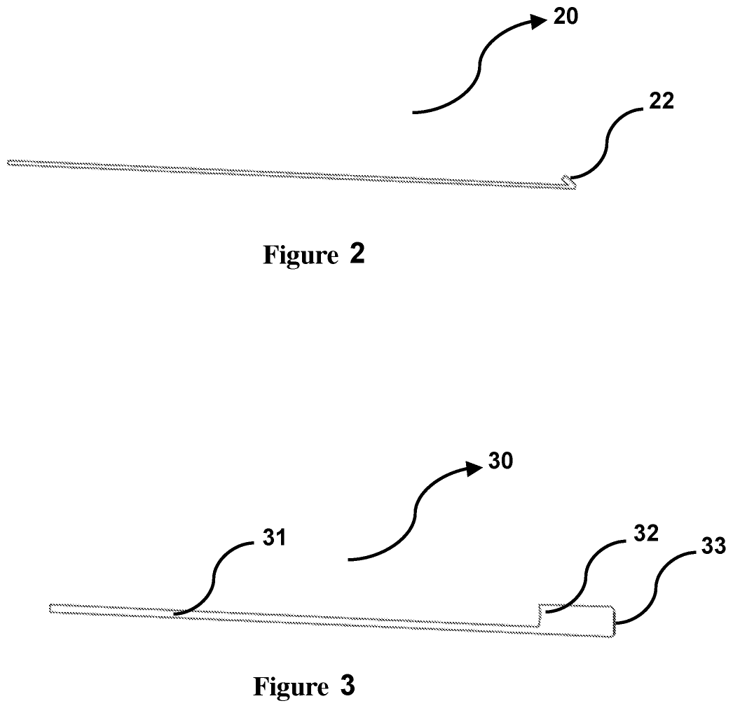

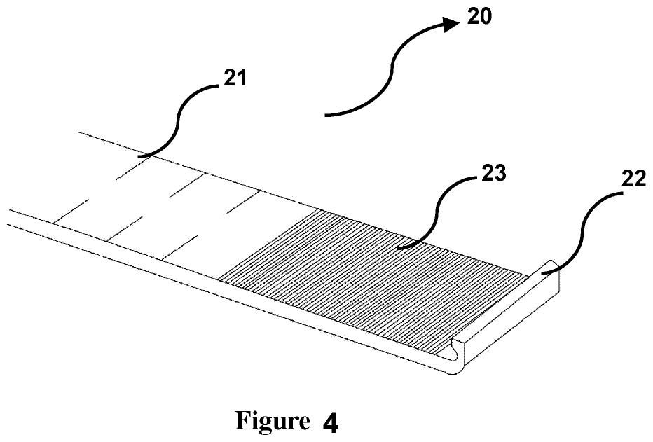

In FIGS. 2 and 4, side and top views of the guide and fascia protecting strip (20) have been shown. The last 1 mm-portion of the strip (20) that has a width of 9-9.5 mm and is made of a material that is rigid enough to allow the strip to move under the fascia at thigh along the whole fascia up to 45 cm, creates a guillotine effect and accordingly it has bent ends suitable to perform a cutting process to free the fascia and rounded out ends (22) that do not damage proximal tissue. A measuring device (ruler) has been provided which has score lines (21) on the strip (20) that is used for measuring the amount of fascia harvested in cms while performing the application. Thus, fascia grafts in any desired lengths can be harvested. This measuring device has an uneven surface (23) starting from 21 centimeters. The uneven surface (23) prevents the clamp (40) which serves to compress the fascia from sliding. If a fascia longer than 21 centimeters is desired to be harvested, the measurement lines that are less discrete on the uneven surface (23) of the guide and fascia protecting strip can be used.

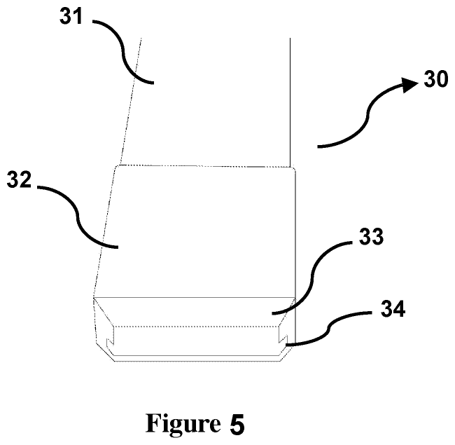

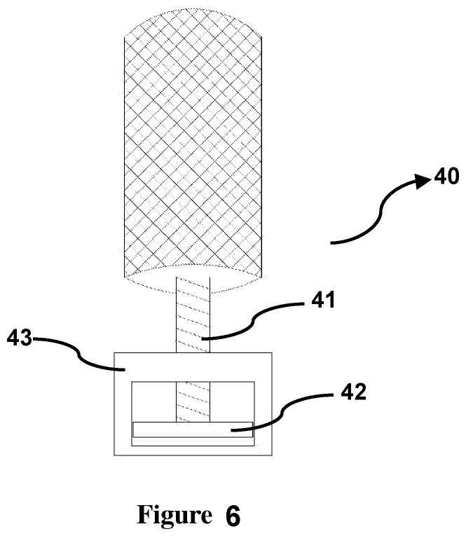

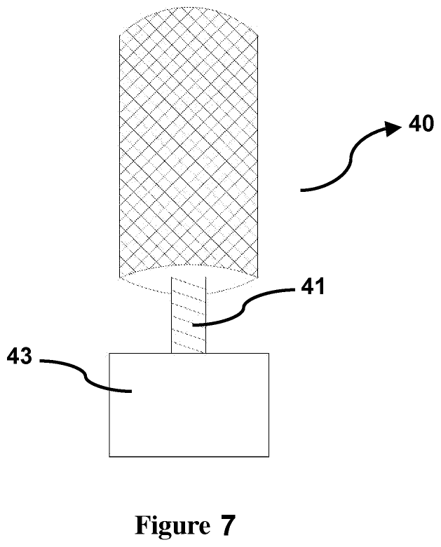

In FIGS. 3 and 5, the side and front view of the fascia cutting tool (30) has been shown. The cutting tool (31), which has the width suitable to allow the guide and protecting strip (20) extending up to 50 cm to pass through, has a separator head (32) on its last 1-2 cm-portion which wraps itself around the fascia and enables the fascia to be separated from proximal tissues. After passing the end portion of the fascia to be harvested which is separated from proximal tissues, through this cutting part, the clamp (40) is used for fixing the fascia on the guide metal strip (20). The clamp (40) both prevents the fascia from sliding by compressing it and helps the metal strip (20) be kept in its position. While the clamp (40) keeps the metal strip (20) and the end of the fascia thereon at a fixed position, the cutting part is pushed to move on the metal strip (20). During this pushing process, as the guide metal strip (20) shall slide in the duct (34) located in the cutting part (31), that is formed specifically for the guide to pass through, the strip shall not damage the fascia. The clamp (40) is formed of a clamp/release bolt (41) having an anti-sliding surface, a foot portion (42) clamping the fascia and a frame (43) allowing the clamp (40) to be positioned in the guide metal strip (20).

The sharpened end portion of the fascia cutting tool (30) comprises a cutting end portion (33) that enables the fascia to be completely released by cutting it with a shearing effect created by the last end portion (22) of the guide and fascia protecting strip (20) when the end of the process is reached after separating the fascia along the whole length of the proximal tissues.

A notch similar to a drawer rail has been formed to prevent the guide strip (20) from shifting while moving inside the cutting tool (31) and from sliding up and down. The area mentioned as a notch is the recesses located on the right and left sides of the duct (34) as shown in FIG. 5. A duct (34) has been provided inside the cutting tool (30) in order to ensure that the guide strip (20) is accurately placed therein and that it can move through it smoothly. During the application, the strip (20) can only move forward and backward without shifting upwards and downwards inside the duct (34). The guide and fascia protecting strip (20) has a length of 40 cm and the fascia cutting tool (30) has a length of 50 cm, which are different in length from each other.

1. A contaminant type detection system, which enables detection of the contaminant types contained by a liquid within an elbow structure enabling a ceramic sanitary ware to be connected to a wastewater installation, the system comprising at least one transmitter/receiver being positioned in a way to face the elbow structure and/or being positioned on the elbow structure and enabling to detection of dielectric properties of the liquid within the elbow structure in the form of phase and/or amplitude by sending wireless signals in a way to pass through the liquid within the elbow structure.

2. A contaminant type detection system according to claim 1, wherein said transmitter/receiver is a transmitter/receiver capable of sending wireless signals in order to detect the contaminant type and capable of catching signals that pass through and are reflected from the elbow structure and/or the liquid within the elbow structure.

3. A contaminant type detection system according to claim 1, comprising at least one first transmitter/receiver capable of sending wireless signals in order to detect the contaminant type and at least one second transmitter/receiver capable of catching the wireless signals, which are sent by the first transmitter/receiver and which pass through the elbow structure and/or the liquid within the elbow structure.

4. A contaminant type detection system according to claim 1, comprising a main processing unit, which determines the signal to be sent by the transmitter/receiver, records the received signal and enables the determination of the contaminant type based on the difference between the sent and received signals.

5. A contaminant type detection system according to claim 4, wherein the main processing unit is a main processing unit, which records the signals sent and received by the transmitter/receiver in the form of amplitude and/or phase.

6. A contaminant type detection system according to claim 1, wherein the transmitter/receiver is a transmitter/receiver operating in the band range of 1 kHz-6 GHz.

7. A contaminant type detection system according to claim 1, wherein the transmitter/receiver is a transmitter/receiver operating in the band range of 6 GHz-18 GHz.

8. A contaminant type detection system according to claim 1, wherein the transmitter/receiver is a transmitter/receiver operating in the band range of 18 GHz-40 GHz.

9. A contaminant type detection system according to claim 1, wherein the transmitter/receiver is a transmitter/receiver operating in the band range of 40 GHz-100 GHz.

10. A contaminant type detection system according to claim 1, comprising at least one display, which enables the user to be informed about the detected contaminant in case an unexpected contaminant is detected in the liquid within the elbow structure.

11. A contaminant type detection system according to claim 1, comprising at least one communication unit, which provides a network connection necessary for informing the user and/or the third parties identified by the user about the detected contaminant and for transmitting the situation information to a database, in case an unexpected contaminant is detected in the liquid within the elbow structure.

12. A contaminant type detection system according to claim 1, wherein the transmitter/receiver is positioned in a way to face the wall of the elbow structure, which enables the ceramic sanitary ware to be connected to the wastewater installation, and/or on the elbow structure, such that it is arranged parallel and/or perpendicular to the floor and/or it is arranged in an angled manner with respect to the floor.

13. A contaminant type detection method, which enables detection of contaminant types contained by a liquid within an elbow structure enabling ceramic sanitary ware to be connected to the wastewater installation, the method comprising the steps of:
sending of wireless signals at certain intervals by a transmitter/receiver to the liquid within the elbow structure and receipt of the sent signals by the same transmitter/receiver and/or another transmitter/receiver (3) depending on use;
comparing the received signals to predefined reference contaminant values and detecting contaminant types and contaminant quantities contained by the liquid within the elbow structure as a result of the comparison; and
informing a user and/or third parties identified by the user about the contaminant and/or saving this information in a database, depending on the choices and preferences, in case a contaminant other than the expected is detected.

14. A contaminant type detection method according to claim 13, wherein in case a contaminant other than the expected is detected in the liquid inside the elbow structure before the ceramic sanitary ware is used by the user, this contaminant is notified and/or saved in the database as a contaminant that is carried with the liquid or that is present in the ceramic sanitary ware.

15. A contaminant type detection method according to claim 13, wherein in case a contaminant other than the expected is encountered in the liquid inside the elbow structure after the ceramic sanitary ware is used by the user, this contaminant is notified and/or saved in the database as a contaminant that originates from the user.

16. A contaminant type detection method according to claim 14, wherein in case a contaminant other than the expected is detected, the user is informed via a display operating in connection with the main processing unit.

17. A contaminant type detection method according to claim 13, wherein in case a contaminant other than the expected is detected, a notification is sent to any device; of the user and/or the persons identified by the user with network connection.

18. A contaminant type detection method according to claim 13, wherein the start and end dates for the menstrual cycle of a user are recorded based on the detection of blood in the liquid inside the elbow structure.

19. A contaminant type detection method according to claim 18, wherein the user is informed about the possibility of pregnancy by way of evaluating, separately or together, the delay in the person's menstrual cycle and/or the variations caused by the contaminant in the amplitude and/or phase of the wireless signals.
